(19) United States
(12) Patent Application Publication
(10) Pub. No.: US 2022/0293226 A1
(43) Pub. Date: Sep. 15, 2022

(54) REAGENT PACK LOAD PLAN OPTIMIZATION METHODS AND SYSTEMS

(71) Applicant: Siemens Healthcare Diagnostics Inc., Tarrytown, NY (US)

(72) Inventors: Ahmet Tuysuzoglu, Jersey City, NJ (US); Yue Zhang, Jersey City, NJ (US); Michael Heydlauf, Cary, NC (US); Luxi Zheng, Cary, NC (US)

(73) Assignee: Siemens Healthcare Diagnostics Inc., Tarrytown, NY (US)

(21) Appl. No.: 17/597,489

(22) PCT Filed: Jun. 10, 2020
(86) PCT No.: PCT/US2020/036922

§ 371 Date: Jan. 7, 2022

Related U.S. Application Data
(60) Provisional application No. 62/877,885, filed on Jul. 24, 2019.
(60) Provisional application No. 63/024,853, filed on May 14, 2020.

(51) Int. Cl. G16H 10/40; G01N 35/00; G16H 40/20

(52) U.S. Cl. CPC G16H 10/40; G01N 35/0092; G16H 40/20; G01N 2035/00673; G01N 2035/0094

(57) ABSTRACT

An optimization method of a diagnostic laboratory system. The method includes receiving, at a system controller, computer-readable data comprising an inventory of a plurality of analyzers included within the diagnostic laboratory system, and types of tests and numbers of the tests to be performed on samples by the diagnostic laboratory system over a planning period; and determining, via a reagent pack optimization module executing on the system controller, a reagent pack loading plan over the planning period. Diagnostic laboratory systems are disclosed, as are other aspects.

### CROSS REFERENCE TO RELATED APPLICATION

This application is related to U.S. Provisional Patent Application No. 62/877,885, entitled “OPTIMIZATION-BASED LOAD PLANNING SYSTEMS AND METHODS FOR LABORATORY ANALYZERS” filed Jul. 24, 2019, the disclosure of which is hereby incorporated by reference in its entirety for all purposes.

### FIELD

This disclosure relates to systems and methods that provide operational planning for a plurality of laboratory analyzers of diagnostic laboratory.

### BACKGROUND

Diagnostic laboratories are currently under financial pressure due to changes in healthcare reimbursements. In particular, healthcare reimbursement rates have dropped significantly over the last few years, and this trend is likely to continue in the future. Taking into consideration that small-scale laboratories have exceedingly low average profit margins, it is highly likely that the trends in reimbursements may render some of these small businesses fiscally nonviable. Further, the stringent reporting requirements can involve information technology infrastructure that many small-scale laboratories have difficulty implementing. These reimbursement reductions coupled with reporting requirements have been a driving force behind centralization and consolidation of such diagnostic testing into larger and larger scale diagnostic laboratories.

Such large scale laboratories process millions of samples per year and they have a significantly higher number of diagnostic instruments connected with automation lines. The operation of such diagnostic laboratories involves consistent and continuous monitoring, evaluation, and intervention by human operators to ensure that results are accurate and that service level agreements are satisfied. Compared to the small-scale laboratories where only a limited number of instruments are utilized, the ability to operate efficiently with minimized operator input is desired.

Accordingly, there is an unmet need to improve operation of large scale diagnostic laboratories including a large number of laboratory analyzers.

### SUMMARY

According to a first embodiment, an optimization method of a diagnostic laboratory system is provided. The method includes receiving, at a system controller, computer-readable data comprising an inventory of a plurality of analyzers included within the diagnostic laboratory system, and types and numbers of tests to be performed on samples by the diagnostic laboratory system over a planning period; and determining, via a reagent pack optimization module executing on the system controller, a reagent pack loading plan over the planning period.

In a further aspect, a diagnostic laboratory system is provided. The diagnostic laboratory system includes a plurality of analyzers that are configured to perform tests on samples, each of the a plurality of analyzers having a fixed menu; and a system controller coupled to the plurality of analyzers, the system controller comprising a reagent pack optimization module having computer executable instructions configured to cause the system controller to generate a reagent pack load plan for the diagnostic laboratory system over a planning period.

Still other aspects, features, and advantages of this disclosure may be readily apparent from the following description and illustration of a number of example embodiments, including the best mode contemplated for carrying out the invention. This disclosure may also be capable of other and different embodiments, and its several details may be modified in various respects, all without departing from the scope of the disclosure. This disclosure is intended to cover all modifications, equivalents, and alternatives falling within the scope of the claims.

### BRIEF DESCRIPTION OF THE DRAWINGS

The drawings, described below, are for illustrative purposes and are not necessarily drawn to scale. Accordingly, the drawings and descriptions are to be regarded as illustrative in nature, and not as restrictive. The drawings are not intended to limit the scope of the invention in any way.

FIG. 1 illustrates a schematic block diagram of a diagnostic laboratory system including a reagent pack optimization module according to one or more embodiments.

FIG. 2A illustrates a schematic diagram of a laboratory analyzer including multiple reagent pack loading spaces according to one or more embodiments.

FIG. 2B illustrates a schematic diagram of an alternate laboratory analyzer having a reagent carousel including multiple reagent pack loading spaces according to one or more embodiments.

FIG. 2C illustrates a schematic diagram of another alternate laboratory analyzer having multiple reagent pack loading spaces according to one or more embodiments.

FIG. 3 illustrates a schematic diagram of an aspiration system including a pipette accessing a reagent pack according to one or more embodiments.

FIG. 4 is flowchart of a method of generating an optimization-based reagent pack load plan for a diagnostic laboratory system according to one or more embodiments.

### DETAILED DESCRIPTION

In large diagnostic laboratory systems including a large number of analyzers, opportunities to increase efficiency can arise when the multiple analyzers have overlapping test menus. A test menu is a menu of tests that the particular analyzer is set up and configured to run as it is currently set up (e.g., existing set up of assay and/or clinical chemistry tests). For example, an analyzer may have capability of running 70 tests, but is currently only configured and set up to run 15 tests. For example, the analyzer may only have reagent packs for the 15 tests only. In particular, there is an unmet need to improve operational efficiency of large-scale diagnostic laboratory systems by providing an optimal allocation of reagent packs across multiple analyzers.

In the present optimization method, the test menus of the various analyzers in the diagnostic laboratory system are fixed over the planning period. By “fixed” it is meant that there will be no introduction of new test types, such as through swapping between analyzers during a particular planning period. This consideration is desirable for many diagnostic laboratory systems, as swapping test types (e.g., assay types) can be quite labor intensive. However, to be clear, the test type (e.g., assay type) makeup can be adjusted at times, such as seasonally, to adjust for any change in the demand for particular test types (e.g., adjustments for higher demand for flu tests in winter season). Thereafter, the present optimization method can be re-employed to establish optimized conditions for a planning period for reagent packs after such a demand adjustment. Thus, the optimization method utilizes any suitable demand data as an input, such as through the use of historical data or through operation of a demand estimation program.

Optimizing the amount of reagent packs to load on the various analyzers can have a significant impact on how efficiently the diagnostic laboratory system can operate. Analyzer as used herein means an device configured to carry out a diagnostic test (hereinafter “test”) of a biological sample (hereinafter “sample”), such as on an immunoassay analyzer, clinical chemistry analyzer, in vitro analyzer, hematology analyzer, molecular analyzer, or the like. Of note, the present optimization method can be re-run frequently (i.e., with high frequency), such as every 8 hours or less, daily or less, weekly or less, monthly or less, in order to adjust the reagent pack placement in the loading spaces with respect to short-term or long term changing test demand trends. The reagent pack loading plan determines optimal placement in available mounting spaces for reagent packs given the number of tests ordered and the type of tests to be run on the plurality of analyzers over the planning period. Furthermore, the present optimization method can addresses the problem of using a subset of the analyzers when there is low test demand, in order to reduce the costs associated with operating an analyzer and yet still cover all the projected or anticipated test orders.

According to the present optimization method, in addition to optimizing the amount of reagent packs to load on the various analyzers, reagent pack optimization module can further optimize for one or more (e.g., multiple) operational efficiency considerations, such as follows:
✓ Operation of the diagnostic laboratory system with a subset of available analyzers,
✓ Load balancing between analyzers,
✓ Reduced turn-around time (TAT),
✓ Efficient reagent usage,
✓ Minimizing quality assurance (QA) costs, and/or
✓ Providing improved diagnostic laboratory system robustness.

Each of the operational efficiency considerations will now be further described.

Operation with a Subset of Instruments: If the test demand can be supported, a diagnostic laboratory system can seek to be operated with a reduced number of analyzers. This can reduce the need for labor hours and can also lower quality control (QC) costs. According to one embodiment of the optimization method, a cost function can be used to explicitly reduce the number of active analyzers (those conducting tests), while the diagnostic laboratory system continues to meet the test demand.

Load Balancing: According to the optimization method, load balancing can be explicitly modeled through a cost function such that all active analyzers perform a substantially similar amount of work. In particular, the cost function can be a combination of one or more balancing types, such as: 1) balancing a number of total tests, 2) balancing a number of a specific test type, 3) time-based balancing, and 4) balancing a number of samples processed. In some embodiments, a combination of more than one balancing types may be used. Imposition of such a load balancing cost function can operate to directly reduce excess wear and can improve turn-around-time (TAT) as any bottlenecks in the diagnostic laboratory system are potentially reduced or minimized.

Reduced Turn-Around Time (TAT): Improving TAT can be one of the top priorities for a diagnostic laboratory system. In particular, better TAT can translate into higher throughput and may allow adherence to service level agreements. Multiple objectives further defined herein can contribute to achieving reduced TAT.

Efficient Reagent Usage: Reagent material is a major cost associated with conducting a diagnostic test (e.g., an assay type), and hence efficient reagent use can be a significant priority for operation of the diagnostic laboratory system.

Quality Assurance (QA) Costs: QA requires labor hours, reagent, and specialized control samples; hence, minimizing QA costs can contribute to reducing overall operational costs for a diagnostic laboratory system. In some embodiments of the optimization method, the method can strive to minimize QA costs by directly counting the unit QA cost to be performed for each test that is deployed.

Improved Laboratory System Robustness: It is desirable to ensure robustness of the diagnostic laboratory system to cover all the ordered tests when one or more analyzers may be off line due to, for example, an analyzer malfunction, analyzer maintenance, or other occurrence that takes the analyzer off line.

Laboratory systems and optimization methods according to embodiments of this disclosure are configured to optimize allocation of reagent packs across multiple mounting spaces of multiple analyzers in the diagnostic laboratory system. The optimization method used may be a mixed integer program, which may be optimized for one or more operational efficiency objectives (see listed operational efficiency considerations above). Further, the optimization method allows for the tailoring of the multiple operational efficiency objectives with respect to the particular needs of a diagnostic laboratory system. In some embodiments, more than one operational efficiency objective may be optimized.

Further, each objective function and constraint defined below can be modified to be applied across a family of analyzers of different types or within a family of analyzers of the same type. Thus, reagent pack optimization module optimizes using one or more optimization objective functions.

### Model Formulation

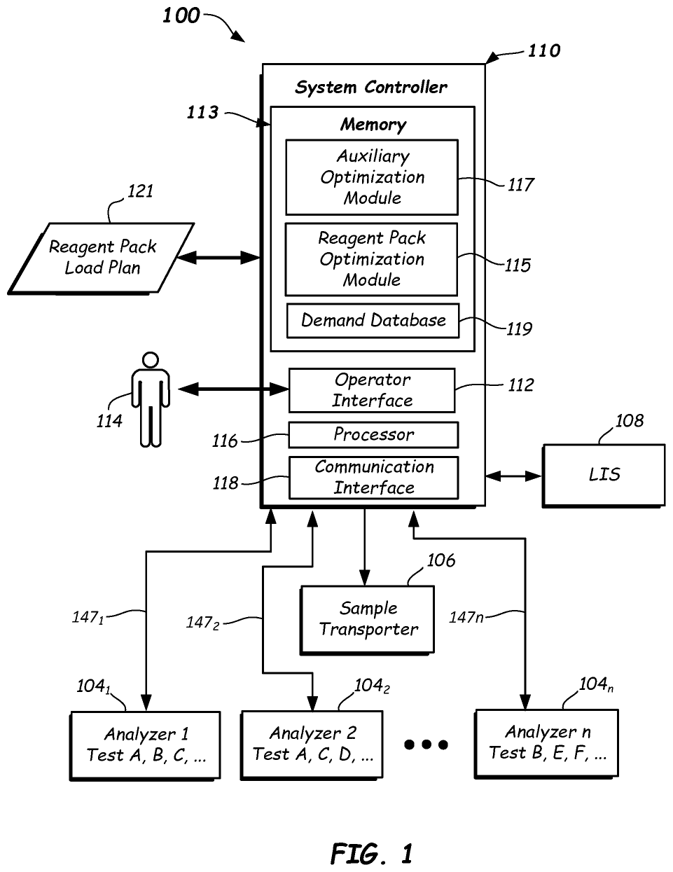

In accordance with aspects of the disclosure, several embodiments of the optimization method are best understood by laying out the notation for the mathematical formulation of the optimization method, as described below. In particular, the optimization methods disclosed herein are useful in providing for an optimal operation of a diagnostic laboratory system 100 including multiple analyzers 104₁-104ₙ, as shown in FIG. 1. An analyzer 104₁-104ₙ can be any inventory-consuming diagnostic analyzer, such as a clinical chemistry analyzer, immunoassay analyzer, in vitro analyzer, hematology analyzer, molecular analyzer, or the like.

Clinical chemistry analyzer as used herein means an analyzer adapted to run assays on samples such as blood serum, plasma, urine, saliva or sputum, cerebrospinal fluid, and the like, to detect the presence of an analyte relating to a disease or a chemical component relating to a drug. Analytes commonly include enzymes, substrates, electrolytes, and specific proteins. Drugs can include drugs of abuse and/or therapeutic drugs. Common clinical chemistry tests includes tests for concentrations of glucose, hemoglobin A1c, sodium, potassium, chloride, lithium, phosphorus, calcium, cholesterol (HDL, LDL), triglyceride, C-reactive protein, bilirubin, lipase, total protein, iron, magnesium, creatinine kinase, urea nitrogen, thyroid stimulating hormone, and the like. Other clinical chemistry tests may be run.

Immunoassay analyzer as used herein means an analyzer adapted to conduct chemical tests used to detect or quantify a specific component in a biological sample using an immunological reaction. Immunoassay analyzers are highly sensitive and specific resulting from the use of antibodies and purified antigens as reagents. Immunoassay analyzers measure the formation of antibody-antigen complexes and detect them via an indicator reaction. High sensitivity is achieved by using an indicator system (e.g., enzyme label) that results in amplification of the measured product. Immunoassays may be qualitative (positive or negative) or quantitative (amount measured). Quantitative immunoassay analyzers measure a signal produced by the indicator reaction.

Immunoassay analyzers can measure (or, in a qualitative assay, detect) an analyte. Immunoassay is a method for measuring analytes present at very low concentrations that cannot be determined accurately by other less expensive tests. Common uses include measurement of drugs, hormones, specific proteins, tumor markers, and markers of cardiac injury, or to detect antigens on infectious agents and antibodies, such as antigens on Hemophilus, Cryptococcus, and Streptococcus organisms in the cerebrospinal fluid (CSF) of meningitis patients. They are also used to detect antigens associated with organisms that are difficult to culture, such as hepatitis B virus and Chlamydia trichomatis, as well as for antibodies produced in viral hepatitis, HIV, and Lyme disease.

Hematology analyzer as used herein means an analyzer adapted to conduct a test on blood, such as a complete blood count (CBC panel), which can include red blood cell (RBC), white blood cell (WBC), hemoglobin concentration, and platelet counts, hematocrit volumes, differential white blood cell counts, red blood cell distribution width, mean corpuscular volume, mean corpuscular hemoglobin, or the like.

Molecular biology analyzer as used herein means an analyzer adapted to conduct molecular biology methods in molecular biology, biochemistry, genetics, and biophysics that involve manipulation and analysis of DNA, RNA, protein, and/or lipid. In particular, molecular biology analyzers are used to analyze biological markers in the genome and proteome, and can be used to detect infectious disease, in oncology, in human leucocyte antigen typing, coagulation, and pharmacogenomics (e.g., a genetic prediction of drugs that may provide effective therapies).

The optimization systems and methods according to embodiments described herein include configurable objective functions and constraints that can be tailored to the unique needs of a particular diagnostic laboratory system. Objective functions may include minimizing QA costs, minimizing unmet capacity cost, maximizing test assignment redundancy, optimizing workload balance, minimizing sample visits, and minimizing total analyzers used, for example.

Constraints may include, e.g., the number of available reagent pack loading spaces of a laboratory analyzer; initial reagent pack volumes; and configured fixed test menus. The optimization systems and methods according to embodiments may be configured to find an optimal assignment of reagent packs based on historical data or the current workload of the lab, and allow selection of a planning period of the laboratory analyzers based on time or the number of samples to be processed. The optimization systems and methods allow easy addition of possible new constraints to an existing diagnostic laboratory system, allow prioritization of objective functions with respect to order of importance, relative normalized weights, or a combination of the two; and/or simulate and observe the effects of various constraints and/or objective function prioritization.

Further details of inventive optimization methods and diagnostic laboratory systems will be described with reference to FIGS. 1-4 herein.

It is assumed that as an input for the present optimization method, details concerning the workload (demand) taking place within the laboratory system 100 such as information about the number of samples and the requested tests on each sample is available, either as 1) an input from the diagnostic laboratory system or LIS or 2) can be predicted via a suitable demand estimation model, such as an artificial intelligence-based model, which may include historical data. This workload (demand) will be referred as the demand input to the diagnostic laboratory system 100. This input can be a subset of a larger payload, such that the optimization method considers only a limited time frame (e.g., a planning period). Planning period is the time period over which the optimization is run, and can be user selectable. Furthermore, the demand input can include more than one optimization options. Multiple optimization options can allow for a better fit to the operating requirements of the diagnostic laboratory system 100, such as relative priority of various objective functions discussed herein.

To better understand the present optimization method, an example architecture of a diagnostic laboratory system 100 is shown and described with reference to FIG. 1. As shown, FIG. 1 illustrates a diagnostic laboratory system 100 according to embodiments that is configured to automatically and efficiently perform tests on a large numbers of biological samples (hereinafter “samples”). In particular, diagnostic laboratory system 100 may include a controller 110, and a large plurality of laboratory analyzers (represented by laboratory analyzers 104₁ through 104ₙ, wherein n is an integer) communicatively coupled to the laboratory analyzers 104₁ through 104ₙ. The number of analyzers 104₁ through 104ₙ can be greater than 1, greater than 3, greater than 6, greater than 10, greater than 20, greater than 50, greater than 100, or even greater than 200 in some embodiments.

In some embodiments, one or more sample transporters 106, such as an automated track or the like, may be used to transport the samples to the various analyzers 104₁ through 104ₙ. The sample transporter 106 may be configured to transport sample containers containing the samples, such as blood collection tubes (not shown) to and from each of the analyzers 104₁ through 104ₙ as well as to and from other locations within the diagnostic laboratory system 100. Sample containers may each be provided with one or more labels that may include identification information thereon, such as, a timestamp, sample identification, requested test(s), patient identification, and/or the like. The label(s) may include, e.g., a barcode and/or have alphanumeric information printed thereon. The identification information may be machine readable at various locations about the diagnostic laboratory system 100, so that the exact location of the sample can be known at all times. The sample transporter 106 may be an automated track such as a railed track (e.g., a mono rail or a multiple rail), a collection of conveyor belts, conveyor chains, moveable platforms, or any other suitable type of conveyance mechanism. Automated track may be circular or have another suitable shape and may be a closed track (e.g., an endless track), and may have one or more offshoots or branches with one or more analyzers (e.g., one or more of analyzers 104₁ through 104ₙ) positioned thereon. Carriers may be part of and may operate on the sample transporter 106 to deliver the samples in the sample containers to the various locations on the sample transporter 106.

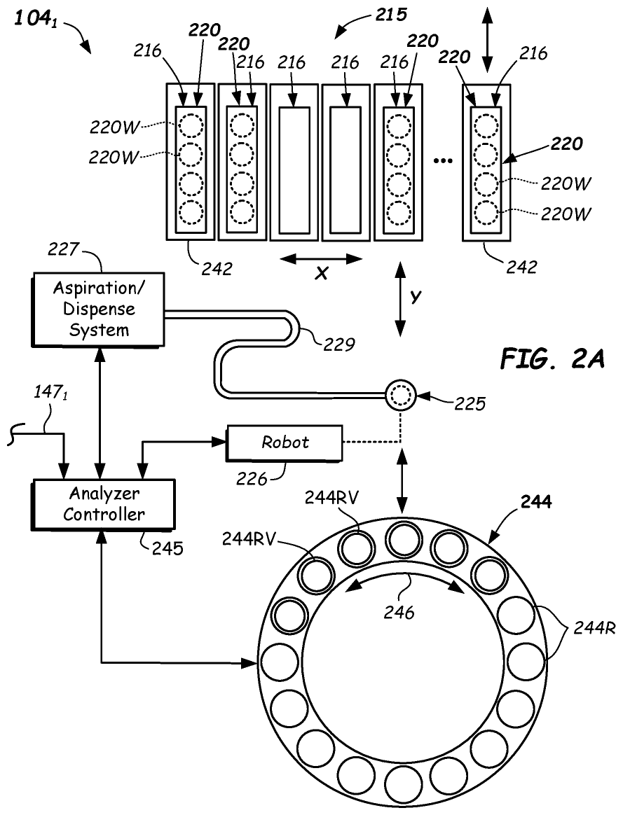

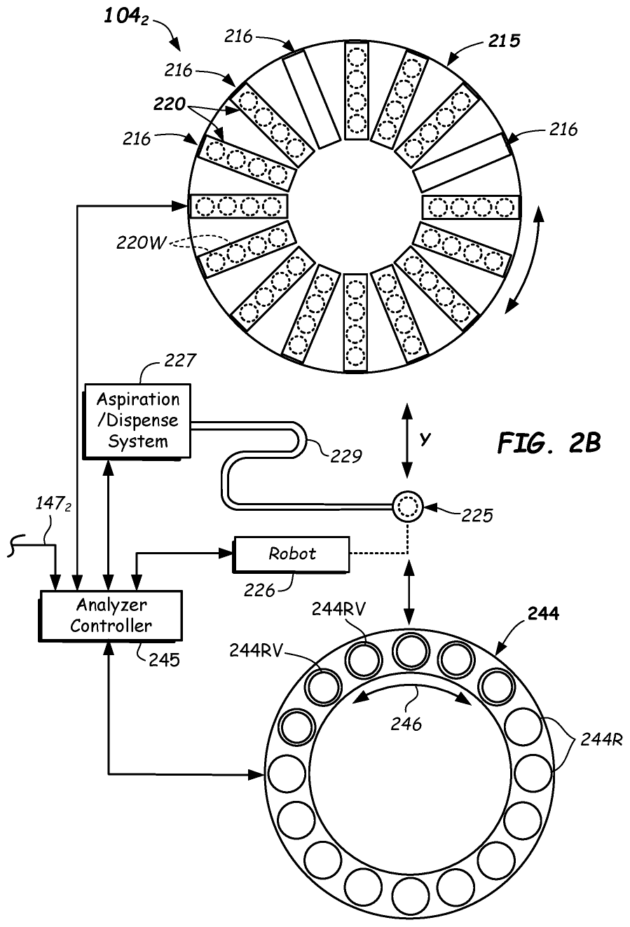

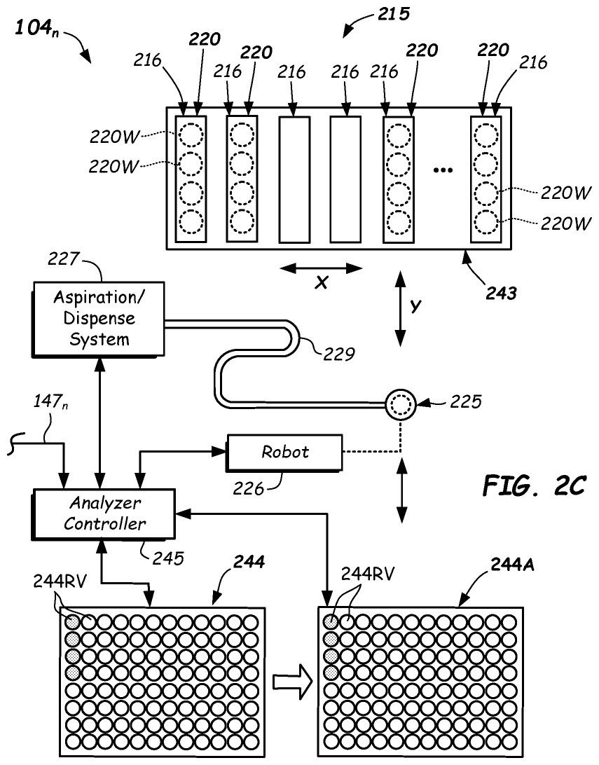

System controller 110 may include an operator interface 112 configured to enable an operator 114 to provide input and intervention to the diagnostic laboratory system 100 when desired. Operator interface 112 may include a user input device (e.g., keyboard—not shown) for entering, e.g., data, requests for status, operational and control commands, etc., to system controller 110. Operator interface 112 may also include a display device (not shown) configured to display status of each of the analyzers 104₁ through 104ₙ, menus, data, and/or messages received from the analyzers 104₁ through 104ₙ. For example, operator interface 112 may provide information about the operation of analyzers 104₁ through 104ₙ, as well as information regarding the status of the tests being performed and that have been performed, status of reagent packs 220 (FIGS. 2A-2C) or line up of tests to be performed thereat.

System controller 110 may control the operation of laboratory analyzer system 100, such as by controlling the sample transporter 106, which ultimately determines the movement and distribution of the samples to the various analyzers 104₁ through 104ₙ, which then carry out various types of tests, as well as the movement elsewhere throughout diagnostic laboratory system 100. System controller 110 may control the operation of various other system components (not shown). Typically, each of the analyzers 104₁ through 104ₙ includes a dedicated computer or workstation therewith (designated as analyzer controller 245 in FIGS. 2A-2C), that can control the specific operation of each of the analyzers 104₁ through 104ₙ. Thus, system controller 110 may interface and communicate with the various analyzer controller 245 by way of communication channels 147₁ through 147ₙ. Communication channels 147₁ through 147ₙ may be part of a communication system enabling data communication between the system controller 110 and the various analyzer controllers 245.

Some functions performed by the optimization method herein may optionally be performed at a computer server that is in digital communication with the system controller 110 over the internet, i.e., they may be cloud based. Thus, system controller 110 may be any suitable computer device or collection of computer devices. However, in such an alternate embodiment, such a cloud server would still functionally be considered part of the system controller 110 and part of the diagnostic laboratory system 100.

As shown, system controller 110 includes a memory 113 (e.g., RAM, ROM, other, or combinations) configured to store programming instructions and other information/data. System controller 110 may also include a processor 116 (e.g., a CPU, microprocessor, or the like) configured to execute programming instructions. System controller 110 may further include a communication interface 118 via which system controller 110 may be coupled to and in electronic communication with LIS 108, sample transporter 106, and analyzers 104₁ through 104ₙ. In some embodiments, communication interface 118 may enable communication over a network (e.g., LAN or WAN). The network may include, e.g., the internet, a local area network (LAN), wide area network (LAN), a wireless local area network (WLAN), a power line communication (PLC) network, or the like. Operator interface 112 may be configured to receive input data to the various operating modules to carry out the optimization.

Diagnostic laboratory system 100 may include other components, equipment, and devices (not shown), such as, e.g., various sensors, barcode readers, robotic mechanisms, sample container loading and/unloading area, pre-processing station (which may include, e.g., an automated centrifuge and sample pre-screening equipment, such as for screening for HIL, or other artifacts such as bubbles, clots, foam), decapper, internet communication device, and the like.

As stated above, each of the analyzers 104₁ through 104ₙ has a fixed test type menu that has been preassigned to it. In some embodiments, some analyzers 104₁ through 104ₙ of the diagnostic laboratory system 100 may be capable of performing the same menu of tests, while others of the analyzers 104₁ through 104ₙ may be capable of performing a different menu of tests, or possibly only a very limited number of tests. The diagnostic laboratory system 100 may be made up of any one or more of an: immunoassay analyzer, clinical chemistry analyzer, in vitro analyzer, hematology analyzer, and molecular analyzer, for example.

As is illustrated in FIGS. 2A-2C, each of the analyzers 104₁ through 104ₙ can contain or have associated therewith some type of reagent pack holder 215 that is configured and adapted to hold one or more reagent packs 220. The reagent pack holder 215 can have a configuration suitable to the particular type of analyzer. Several different types of reagent pack holders 215 are shown in FIGS. 2A-2C, such as a slot-type holder (FIG. 2A), a reagent carousel type holder (FIG. 2B), or a tray type holder (FIG. 2C). Each reagent pack holder 215 can include multiple mounting locations referred to herein as mounting spaces 216 (a few labeled) that are configured to receive a reagent pack 220 thereat. Mounting spaces 216 may have any suitable configuration designed to receive a reagent pack 220, and may be a slot, recess, or groove, or any other suitable mounting structure, and may include a retention feature helping to secure and retain the reagent pack 220 in place. The reagent packs 220 can have any suitable structure and construction that is applicable to the particular analyzer 104₁ through 104ₙ they are used with. There may be various types of reagent packs 220 that can be loaded onto the mounting spaces 216 and they may have one reagent therein, such as the same reagent in all reservoirs thereof, or any number of reagents or other liquids therein. The number, location, and mix of types of reagent packs 220 to be placed on each analyzer 104₁ to 104ₙ is determined by the optimization method carried out by the reagent pack optimization module 115 disclosed herein.

Further, the number of mounting spaces 216 in each of the analyzers 104₁ through 104ₙ, as well as the type or configuration of the reagent pack holder 215 can differ across the various analyzers 104₁ through 104ₙ in the diagnostic laboratory system 100. The analyzer 104₁ through 104ₙ may further include more conventional components than are illustrated in FIGS. 2A-2C, such as heater(s), wash station(s), cuvette and pipette tip loaders, pipette wash stations, additional pipettes, waste receptacles, optical emission reader(s) for determining concentration levels of an analyte or constituent, and other conventional components not shown.

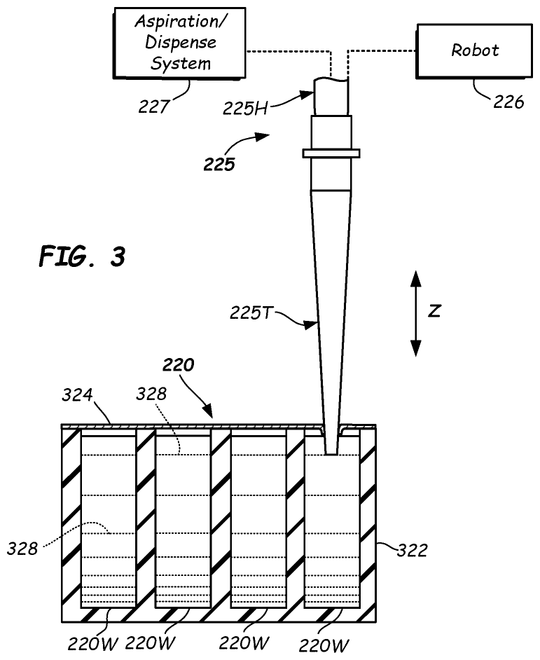

In further detail as shown in FIGS. 2A-2C and FIG. 3, in some embodiments each of the reagent packs 220 may include one or more wells 220W. Covers 324 may be sealed over the one or more wells 220W, and may be punctured and/or accessed by an automated pipette 225 via Z axis motion provided by a robot 226. Pipette 225 may include a detachable pipette tip 225T that can be detached from a pipette head 225H and discarded after a use, to minimize cross contamination. In some embodiments, the reagent packs 220 may be identical to all reagent packs 220 loaded onto the in the reagent pack holder 215 (FIGS. 2A-2B), or optionally, at least some of the reagent packs 220 may have a different configuration, such as including more or less numbers of wells 220W, a different size or shape configuration, and containing a different reagent than the others. For example, some reagent pack holder 215 can hold reagent packs 220 containing ancillary reagents that may include a different shape. To be clear, however, reagent pack 220 does not include containers that contain bulk acid reagent or bulk base reagent, diluents, and/or buffer, suspensions of magnetic particles that are used on every test. These types of containers are filled as needed by the operator 114 as they are used for virtually all tests.

The reagent pack 220 may include a reagent pack body 322, formed from a plastic material, for example, and a plurality of wells 220W formed in the reagent pack body 322. Each well 220W in the reagent pack body 322 may include an open top and a closed bottom. In the embodiment depicted in FIG. 3, the reagent pack 220 includes four wells. However, other embodiments of the reagent pack 220 may include more or fewer than four wells.

The wells 220W may contain liquids 328, such as one or more reagents, one or more ancillary reagents, and/or one or more allergens. However, the wells 220W may contain other liquids. A reagent pack 220 may contain some or all the reagents and/or other liquids needed for a particular type of test (assay). Some of the various reagents may be the same or different.

Now referring to FIG. 2A, the analyzer 104₁ shown may include a reagent pack holder 215 configured to receive a plurality of reagent packs 220 in mounting spaces 216 thereof. The reagent pack holder 215 can be configured to have a plurality of slides 242 having the mounting spaces 216 disposed thereon. The slides 242 may be configured to side in the Y direction (orthogonal to the Z direction (FIG. 3) relative to a frame or other structure of the analyzer 104₁, and may be provided in a refrigerated area of the analyzer 104₁ in some embodiments. Each of the slides 242 may slide laterally in the Y direction a sufficient amount to expose the mounting space 216, such that a reagent pack 220 may be received therein if called for by the reagent pack load plan.

The analyzer 104₁ may further include an incubation member 244 that may include a plurality of receptacles 244R therein that are configured to support and/or receive a plurality of reaction vessels 244RV therein. The reaction vessels 244RV may be configured to contain at least biological samples acquired from patients and reagents and/or other liquids from the reagent packs 220, and possibly other liquids. In some embodiments, the reaction vessels 244RV can be cuvettes. In some embodiments, the incubation member 244 may be provided in the form of a sample carousel, which may be an incubation ring carousel or other type of carousel that incubates and otherwise prepares the processed samples for testing. Both the reagent pack holder 215 and the incubation member 244 may include electromechanical devices (e.g., motors—not shown) that cause motion thereof. For example, the reagent pack holder 215 may move back and forth in the X direction and the incubation member 244 may rotate (as indicated by arrow 246). The incubation member 244 may also be heated to a predetermined temperature. Both may be electrically coupled to an analyzer controller 245 that generates signals to operate the electromagnetic devices and other system components thereof. Analyzer controller 245 further can electronically communicate with the system controller 110, as indicated by communication line 147₁.

The analyzer 104₁ may further include the robot 226 that is configured to transport the pipette 225 between wells 220W in the reagent packs 220 and the reaction vessels 244RV in the incubation member 244. The robot 226 may include any suitable configuration that is configured to move the pipette 225 between the reagent packs 220 located in the reagent pack holder 215 and the incubation member 244. In some embodiments, the robot 226 is coupled to and is configured to move the pipette 225 in the Y, and Z (into and out of the paper in FIG. 2A). The reagent pack holder 215 may, in some embodiment, be moveable in the X direction by any suitable means to enable any of the reagent packs 220 populated to be accessed. The robot 226 may be electrically coupled to the analyzer controller 245, which may generate signals to operate the robot 226.

The analyzer 104₁ may further include an aspiration/dispense system 227 that may be coupled to the pipette 225, such as by a conduit 229. The aspiration/dispense system 227 may control amounts of liquids aspirated and dispensed from the reagent pack 220 for a particular test. The aspiration/dispense system 227 may be electrically coupled to the analyzer controller 245, which controls one or more pumps responsive to one or more sensors (not shown) and the like to perform the aspiration and dispensing. Optionally, the reagent pack holder 215 may be immoveable in the X direction and the robot 226 may include X, Y and Z axis motion capability enabling any of the wells 220W of the various reagent packs 220 to be accessed.

FIG. 2B illustrates another example embodiment of analyzer 104₂ of the diagnostic laboratory system 100 wherein the reagent pack holder 215 can be a carousel configured to have a plurality of mounting spaces 216 radially disposed thereon. The mounting spaces 216 are each configured to receive a reagent pack 220 thereat. Each reagent pack 220 may include one or more wells 220W formed therein containing one or more reagents or other liquids used to carrying out a specific test. As will be apparent from the following, in an optimized diagnostic laboratory system 100, not all of the mounting spaces 216 will include a reagent pack 220. For example, some of the mounting spaces 216 may be empty.

The analyzer 104₂ may further include an aspiration/dispense system 227 and pipette 225 as previously described for FIG. 2A. As shown, the reagent pack holder 215 may be moveable in rotation in one or more rotational directions by a suitable motor and drive (not shown). The robot 226 may include Y axis and Z axis motion capability enabling any of the wells 220W of the various reagent packs 220 to be accessed by the pipette 225 for aspiration of reagent or other liquid therefrom and delivery and dispense to a reaction vessel 244RV provided in the incubation member 244. Incubation member 244 can be identical to that described in the embodiment of FIG. 2A and is conventional.

Now referring to FIG. 2C, the analyzer 104ₙ may include a reagent pack holder 215 configured to receive a plurality of reagent packs 220 in mounting spaces 216 of a tray 243. In some embodiments, some or all of the tray 243 may be provided in a refrigerated area of the analyzer 104ₙ. The analyzer 104ₙ may further include incubation members 244, 244A that may include a plurality of reaction vessels 244RV therein. For example, the reaction vessels 244RV may be provided on a 96 well test plates wherein each respective well can comprise a reaction vessel 244RV. The reaction vessels 244RV can be configured to contain at a least biological sample acquired from a patient or extracted components thereof together with reagents and/or other liquids dispensed from the reagent packs 220.

In this embodiment, the biological sample (s) have been pre-processed on an extraction plate as the incubation member 244 to provide eluate containing the sample DNA or RNA, for example. Thus, in this embodiment, the incubation member 244 may be provided in the form of a 96 well test plate that incubates and otherwise prepares the DNA templates for replication and testing. Once the DNA or RNA are extracted, the eluate may be transferred and replicated on a second incubation member 244A. The incubation member 244, 244A may include electromechanical devices (e.g., agitators—not shown) that can cause motion thereof. For example, one or both of the incubation members 244, 244A may move back and forth at various times to promote mixing. One or both of the incubation members 244, 244A may also be heated at times to a predetermined temperature and may further undergo multiple heating and cooling cycles as are known to those of skill in the art. One or more components of the incubation members 244, 244A and other system components may be electrically coupled to an analyzer controller 245, which generates signals to operate the electrical devices (e.g., heaters, robot 226, aspiration/dispense system 227, mixers, etc.) and other system components thereof. Analyzer controller 245 further can electronically communicate with the system controller 110, as indicated by communication line 147n.

The analyzer 104ₙ may further include a robot 226 that is configured to transport a pipette 225 between wells 220W in the various reagent packs 220, as required for the various processes, and the reaction vessels 244RV in the incubation members 244, 244A, as needed, to carry out the DNA template extraction and replication. The robot 226 may include any suitable configuration that is configured to move the pipette 225 between the reagent pack holder 215 and the incubation members 244, 244A. In some embodiments, the robot 226 can be configured to move the pipette 225 in the X, Y, and Z (into and out of the paper in FIG. 2C). Thus, the reagent pack holder 215 comprising the tray 243 of populated reagent packs 220 can be accessed by the pipette 225.

Similar to the other embodiments, the analyzer 104ₙ may further include an aspiration/dispense system 227 that may be coupled to the pipette 225, such as by a conduit 229. The aspiration/dispense system 227 may control amounts of reagents and other liquids aspirated and dispensed from a reagent pack 220 to the incubation member 244, 244A for conducting a particular test (e.g., assay). The aspiration/dispense system 227 may be electrically coupled to the analyzer controller 245, which controls one or more pumps responsive to one or more sensors (not shown) and the like to perform the aspiration and dispensing.

For each of the analyzers 104₁ through analyzer 104ₙ, each of mounting spaces 216 of the reagent pack holder 215 may receive a reagent pack 220 including a same reagent or a different reagents. Moreover, each mounting space 216 may include different reagents therein. The number of mounting spaces 216 in each of the reagent pack holders 215 may also differ among the analyzers 104₁ through analyzer 104ₙ

### Optimization Method

Now referring to FIG. 1 through FIG. 4, the optimization method will be described. For each of the analyzers 104₁ through 104ₙ in the diagnostic laboratory system 100 there are a number of mounting spaces 216 available. Given the fixed menus for each of the analyzers 104₁ through 104ₙ, the present optimization method can, using the reagent pack optimization module 115, determine an optimal placement in the available mounting spaces 216 for the reagent packs 220 given the number of tests that have been ordered and the type of tests to be run on the analyzers 104₁ to 104ₙ over the planning period.

According to the optimization method, first, let nᵢⱼ denote the non-negative integer variable indicating the number of tests j ∈ [symbol] to run on the analyzers i ∈ [symbol] ,

wherein

[symbol] represent the sets of tests, and

[symbol] represent the analyzers.

So then:

$${n}_{i j} = \{ \begin{matrix}{n}_{i j} ∈ {ℤ}^{+} , & if instrument i is configured / allowed to run test j , \\ 0 , & otherwise\end{matrix} .$$

When nᵢⱼ is zero, the reagent pack 220 corresponding to test j does not need to be loaded in a mounting space 216 on analyzer i as this particular analyzer i (e.g., should not run any of these tests). In the case when all nᵢⱼ for analyzer i is zero, it does not need to be run.

Further, according to the optimization method xᵢⱼ is defined to denote a binary payload variable, which indicates the current distribution of tests, j ∈ [symbol] , across the analyzers, i ∈ [symbol] :

$${x}_{i j} = \{ \begin{matrix}1 , & if instrument i is configured to run test j , \\ 0 , & otherwise\end{matrix} .$$

As mentioned earlier, the distribution of the tests (test menus) for each of the analyzers 104₁ through 104ₙ is assumed to be fixed.

In this optimization method, we consider the demand of the diagnostic laboratory system 100, that is the breakdown of tests by test type and number of tests over the planning period is considered to be provided or predicted by an auxiliary optimization model 117, which may be a machine learning model or any suitable model or software that otherwise estimates demand over the planning period, for example by using historical data over similar timeframes. Once the demand has been estimated over the planning period, it is stored in demand database 119. The optimization method then defines as a binary matrix:

S ∈ [symbol] encoding the ordered tests for each sample as:

$${S}_{a j} = \{ \begin{matrix}1 , & if sample a requires test j , \\ 0 , & other wise\end{matrix} .$$

where α ∈ [symbol] , j ∈ [symbol] , and [symbol] is the set of all samples.

Given the sample data S, and the current distribution of fixed test menus across the analyzers 104₁ to 104ₙ, the optimization method aims to find a reagent pack load plan 121 that in the depicted embodiment may correspond to solving a mixed integer program that optimizes functional objectives under equipment-related constraints and testing-related constraints. Table 1 below lists all the related variables for quick reference.

TABLE 1
Variable list involved in the optimization method

| Variable | | |
|---|---|---|
| Name | Definition | Type |
| nᵢⱼ | Total number of test j that will be processed on analyzer i | Optimization |
| xᵢⱼ | 1 if analyzer i is configured to perform test j | Payload |
| ϵⱼ | Slack variable indicating the total number of | Optimization |
| | uncompleted test j due to unmet capacity | |
| Iₐᵢ | 1 if analyzer i can be used to cover a test or tests | Optimization |
| | requested by sample a | |
| rⱼ | Volume of consumed reagent for performing one test j | Payload |
| | (analyzer independent) | |
| qⱼ | Unified quality assurance related costs of test j | Payload |
| mⱼ | Minimum number of analyzers to perform test j on | Payload |
| REⱼ | Redundancy factor of test j indicating the relative need | Payload |
| | to run test j on multiple analyzers | |
| Vᵢⱼ | Total volume of the reagent recurrently loaded in | Payload |
| | analyzer i for test j during current planning period | |
| Sₐⱼ | 1 if sample a requires test j | Payload |
| npᵢⱼ | Number of tests j that can be performed per reagent | Payload |
| | pack 220 on analyzer i | |
| tⱼ | Amount of time it takes to perform test j | Payload |
| Mᵢ | Maximum throughput of analyzer i during the | Payload |
| | optimization planning period | |

Thus, according to the optimization method, the objective is to:

minimizef(nᵢⱼ)

subject to:g(nᵢⱼ,xᵢⱼ,S)≥0,nᵢⱼ∈ [symbol] ,

where f and g are composite functions of objectives and constraints, respectively.

### Optimization Objectives

According to the optimization method, the following provides a mathematical formulation of optimization objectives (targets). According to the optimization method, an indicator function I is define as:

I: [symbol] ₊^{N×J}→{0,1}^{N×J}

Here [symbol] ₊ refers to the set of all non-negative integers. For any matrix M ∈ [symbol] ₊^{N×J}, then:

I(M)ᵢⱼ=1ifMᵢⱼ>0, and

I(M)ᵢⱼ=0otherwise

The present optimization method utilizes demand data as an input or optionally output from an auxiliary optimization module 117, which may be an artificial intelligence-based prediction model of demand. Continuity across the planning period is accomplished through adherence to the test distribution xᵢⱼ.

In particular, as is shown schematically in FIG. 1, in the operation of the diagnostic laboratory system 100, the assignments of tests to the analyzers 104₁ to 104ₙ can be achieved by an auxiliary optimization module 117, such as a seasonal solution engine, which can optimize the fixed menus for the analyzers 104₁ to 104ₙ based upon the expected demand for test type and test numbers thereof over the planning period. The present optimization method then can be used, on a regular basis such as every 8 hours or less, daily or less, weekly or less, or even monthly or less for determining an optimized allocation and placement of amounts of reagent packs 220 to the various analyzers 104₁ to 104ₙ having such fixed menus. Other suitable periods for running the optimization method may be used.

The following provides a detailed discussion of the mathematical formulations of the various optimization objectives that may be used. One or more optimization objectives can be used by the optimization method, such as:
minimizing QA costs,
minimizing unmet capacity cost,
maximizing test assignment redundancy,
optimizing workload balance,
minimizing sample visits, and
minimizing total analyzers used.
   Each objective function will now be described more fully below.

1) Minimize QA Costs: A first optimization objective functions to minimize quality assurance (QA) costs. This objective considers total cost associated with quality control (QC) material, reagent cost used in the QC process, and costs associated with downtime as follows:

[symbol]

_{QA}= [symbol] Σ_{j∈τ}qⱼI(nᵢⱼ),

where qⱼ is the unit QA cost. The QA cost for test j is only incurred when test j has to be processed on analyzer, such as nᵢⱼ>0.

2) Minimize Unmet Capacity Cost: A second objective function operates to minimize unmet capacity cost. In particular, the test demand during the planning period of the optimization method can be readily available or can be predicted with the auxiliary optimization module 117. Given the amount of resources, such as the number of analyzers and optimization time frame (planning period), the capacity of the diagnostic laboratory system 100 might not be sufficient to process all the samples. Thus, the method can denote the number of uncompleted tests j as ∈ⱼ and then state the unmet capacity cost as follows:

[symbol]

_{MC}= [symbol] ∈ⱼ

An optimality condition for this second objective is:

[symbol]

∈ⱼ=0

meaning all the tests can be processed within the given resources.

3) Maximize Test Assignment Redundancy: A third objective function can operate to maximize test assignment redundancy. It is often needed that certain tests should be deployed on more than a single analyzer due to robustness or issues with uncertain demand. Thus, the third objective function can associate a redundancy factor REⱼ≥0 for each test j. A large redundancy factor REⱼ means it may be desirable to have test j capable of being performed on multiple analyzers (e.g., more than one of the analyzers 104₁ to 104ₙ). This is a payload variable and can be tailored with respect to the needs of diagnostic laboratory system 100. The following third objective function can be maximized to achieve redundancy:

[symbol]

_{R}= [symbol] REⱼ [symbol] I(nᵢⱼ)

This third objective function simply counts the number of analyzers (from analyzers 104₁ to 104ₙ) that each test is deployed on and accumulates a total redundancy factor.

4) Optimize Workload Balance: The fourth objective function operates to optimize workload balance. Improving the workload balance of analyzers 104₁ to 104ₙ with fixed menus can help reduce excess wear and improve turn-around time (TAT) as bottlenecks may be potentially eliminated. The method can explicitly model load balancing through a cost function that can incorporate three different strategies. However, each of these different strategies has their own merit and the present optimization objective function can allow the use any combination or subset of the strategies. The workload balance strategies comprise:

i) Operation-time balancing: Operation-time balancing strives for equal processing times across all analyzers 104₁ to 104ₙ. This strategy operates to account for the fact that certain tests can take longer and accumulation of such tests to specific analyzers 104₁ to 104ₙ can create bottlenecks especially when there are multiple types of analyzers families that may be operated together.

ii) Test-type balancing: The workload of each test should be distributed equally across the analyzers 104₁ to 104ₙ the particular test is deployed on. This strategy enforces balancing within a family of like analyzers 104₁ to 104ₙ.

iii) Total workload balancing: Total number of tests to be performed should be balanced across all the analyzers 104₁ to 104ₙ. This cost function favors equal distribution of the total test load both across different analyzer families and within analyzer families.

iv) Samples balancing: Samples balancing strives for equal number of samples to be processed across all analyzers 104₁ to 104ₙ. This cost function favors equal distribution of the samples both across different analyzer families and within analyzer families.

To achieve equal processing times across analyzers 104₁ to 104ₙ, the optimization method can measure and penalize any deviation from an average processing time. This average processing time is defined as follows:

$$\overline{t} = \frac{{\sum }_{j ∈ 𝒯} {t}_{j} {\sum }_{a ∈ 𝒜} {S}_{aj}}{\vert ℳ \vert}$$

where tⱼ is the time it takes to perform one sample of test j.

The method can then minimize the following cost for time-balancing:

$${𝒞}_{LB , T} = {\sum }_{i ∈ ℳ} {( \overline{t} - {\sum }_{j ∈ 𝒯} {t}_{j} {n}_{i j} )}^{2}$$

This cost quadratically penalizes the deviation of the total test time an analyzer (e.g., any of analyzers 104₁ to 104ₙ) would take to process its samples from the average t̅.

In balancing test-type workload, the method can penalize the deviation of nᵢⱼ, assigned number of test js on analyzer i, from a nominal value. This nominal value for each test can be provided as the following average:

$${\hat{n}}_{j}^{a v g} = \frac{{\sum }_{a ∈ 𝒜} {S}_{a j}}{{\hat{N}}_{j}}$$

where \hat{N}ⱼ is the number of analyzers 104₁ to 104ₙ that has test j assigned thereto. However, the computation of this nominal value requires knowledge of \hat{N}ⱼ, which can only be obtained by solving another optimization problem. To untangle this dependence, the method can use a surrogate, Nⱼ, defined as a number of analyzers 104₁ to 104ₙ that has test j enabled on its fixed test menu. Since \hat{N}ⱼ≤Nⱼ, we obtain a lower bound to the nominal value, nⱼ^{avg}, such that nⱼ^{avg}≤\hat{n}ⱼ^{avg}.

This objective function can then be written as follows:

[symbol]

_{LB,A}= [symbol] [symbol] (nⱼ^{avg}−nᵢⱼ)².

Total workload balancing can also be enforced by minimizing the deviation from a nominal value. In this case the nominal value is defined as follows:

$${\hat{n}}^{avg} = \frac{{\sum }_{i ∈ ℳ} {\sum }_{j ∈ 𝒯} {n}_{i j}}{\vert ℳ \vert}$$

However as nᵢⱼ are optimization variables and hence the nominal value is not known in advance, instead, the method uses a surrogate:

$${n}^{a v g} = \frac{{\sum }_{a ∈ 𝒜} {\sum }_{j ∈ 𝒯} {S}_{a j}}{\vert ℳ \vert}$$

This surrogate is an upper bound, such that \hat{n}^{avg}≤n^{avg}, as not all the tests might be completed with the available analyzers 104₁ to 104ₙ. The objective function is then:

$${𝒞}_{LB , S} = {\sum }_{i ∈ ℳ} {( {n}^{a v g} - {\sum }_{j ∈ 𝒯} {n}_{i j} )}^{2} .$$

In balancing sample workload, the method can force a number of samples to be loaded on the analyzers 104₁ through 104ₙ to be close to uniformly distributed, by penalizing the deviation of number of samples loaded on each analyzer from a theoretical average. The objective function can be written as follows:

$${𝒞}_{LB , SL} = {\sum }_{i ∈ ℳ} {( {\sum }_{a ∈ 𝒜} {I}_{a i} - \frac{\vert 𝒜 \vert}{\vert ℳ \vert} )}^{2}$$

where Iₐᵢ is a binary variable indicating whether sample a will require analyzer i:

$${I}_{a i} = \{ \begin{matrix}1 , & if sample a requires instrument i , \\ 0 , & otherwise\end{matrix} .$$

| [symbol] | and | [symbol] | are the size of the set [symbol] and [symbol] , corresponding to the total number of samples and total number of analyzers, respectively. Minimizing this sample balancing objective function can encourage each analyzer 104₁ through 104ₙ to analyze a similar amount of samples.

When more than one workload balancing costs are used, then the overall workload balancing objective can be written as follows:

[symbol]

_{LB}=β₁ [symbol] _{LB,T}+β₂ [symbol] _{LB,A}+β₃ [symbol] _{LB,S}+β₄ [symbol] _{LB,SL},

where β₁, β₂, β₃, and β₄ are non-negative weights adjusting a relative contribution of each balancing strategy.

Unlike the previous objective functions, the workload balancing cost function is quadratic. Inclusion of such objectives to the method significantly increases the computational burden as the problem becomes an instance of mixed integer quadratic programming. This relatively high computational burden can be overcome by measuring and minimizing the linear deviation from the nominal values through the use of integer non-negative slack variables for all three cost functions that make up [symbol] _{LB}. In linearizing [symbol] _{LB,A} we use n₊^{ij} and n₋^{ij} as slack variables corresponding to the excess and missing load of test j on analyzer i:

minimize [symbol] [symbol] (n₋^{ij}+n₊^{ij})

subject tonᵢⱼ=n_{avg}^{j}+n₊^{ij}−n₋^{ij},n₊^{ij}≥0,n₋^{ij}≥0.

Linearization of [symbol] _{LB,T}, [symbol] _{LB,S} and [symbol] _{LB,SL} follows a similar approach with the introduction of slack variables and can be readily determined.

5) Minimize Sample Visits: A fifth objective function can be used to minimize total analyzer visits to be made by the samples: Each sample in the workload generally requires visits to multiple ones of the analyzers 104₁ to 104ₙ. This is due to the test menu differences on the same types of analyzers 104₁ to 104ₙ, or the need to visit different types of analyzers 104₁ to 104ₙ. Each such analyzer visit of a sample affects the sample's TAT along with the overall TAT. The method can account for this phenomenon by counting a number of total stops samples are required to make, as given by:

$${𝒞}_{s t o p s} = {\sum }_{a ∈ 𝒜} {\sum }_{i ∈ ℳ} {I}_{a i}$$

where Iₐᵢ is a binary variable indicating whether sample a will require analyzer i:

$${I}_{a i} = \{ \begin{matrix}1 , & if sample a requires instrument i , \\ 0 , & othe r w ise\end{matrix} .$$

Minimizing this objective will encourage sample a to request as few analyzers 104₁ to 104ₙ as possible, and thus reduces the number of stops a sample makes, directly optimizing TAT.

6) Minimize Total Analyzers Used: A sixth objective function is to minimize the total number of analyzers 104₁ to 104ₙ to be used during the planning period: The test demand of the diagnostic laboratory system 100 can fluctuate due to many factors, such time of the day, day of the week, or time of the year. In times of low test demand, running all the analyzers 104₁-104ₙ can be unnecessary, such as when a subset of the analyzers 104₁ to 104ₙ can handle the test demand (workload). Such an objective can reduce the need for labor, reduce quality control costs, and reduce reagent costs. The optimization method can incorporate this objective through the following cost function:

[symbol]

ᵢₙₛₜ= [symbol] I( [symbol] nᵢⱼ)

The expression I( [symbol] nᵢⱼ) is an indicator if analyzer i is processing any tests. A cost is not incurred for analyzer i only if nᵢⱼ=0 for all assays j ∈ [symbol] .

### Optimization Constraints

According to the method, mathematical formulations of various optimization constraints are provided. These optimization constraints make sure that for the allocation of resources that the optimization method generates is implementable and continuity of test types in the analyzers 104₁-104ₙ is guaranteed. They can be classified into three categories: 1) a menu feasibility constraint, 2) a capacity constraint, and 3) a workflow continuity constraint, as outlined below.

1) Menu Feasibility Constraints: These feasibility constraints ensure that the analyzers 104₁-104ₙ load reagent packs 220 for all the types of tests that are in the test demand (payload).

2) Capacity Constraints: These capacity constraints arise due to the physical limitations of the diagnostic laboratory system 100, such as the number of analyzers 104₁-104ₙ, analyzer throughput, as well as the quantity of available reagent packs 220 and mounting spaces 216.

3) Workflow Continuity Constraints: These continuity constraints ensure the continuity of tests on the analyzers 104₁-104ₙ such that tests are not swapped during the optimization. These constitute a part of the optimization method facilitating the high-frequency optimization without increasing the need for manual labor.

The mathematical formulation of each optimization constraint can be as follows:

1) Mounting Spaces Constraints: The number of reagent packs 220 to load on an analyzer 104₁-104ₙ cannot be greater than the number of mounting spaces 216:

$$0 \leq {\sum }_{j ∈ 𝒯} ceil ( \frac{{n}_{i j}}{n {p}_{i j}} ) \leq \vert 𝒲 \vert , ∀ i ∈ ℳ .$$

Here ceil is a standard ceiling function, which returns the smallest integer value that is greater than or equal to the input number npᵢⱼ is the number of tests j that can be performed per reagent pack 220. We note that npᵢⱼ also depends on the analyzer i. This notion is used to accommodate scenarios where different analyzers 104₁-104ₙ can load reagent packs 220 having a different size for the same test.

2) Loaded Reagent Pack Constraints: The types of loaded reagent packs 220 should cover all the ordered tests for the samples. This constraint ensures that for each requested test, there is at least one analyzer 104₁-104ₙ to perform it:

I( [symbol] Sₐⱼ)≤I( [symbol] nᵢⱼ),∀j∈ [symbol] .

This loaded reagent pack constraint induces J inequalities. Given a fixed test type j, I( [symbol] Sₐⱼ) ∈ {0,1} is a binary variable indicating whether test j is ordered for the samples. The right-hand side of this constraint, I( [symbol] nᵢⱼ) ∈ {0,1}, indicates whether there exists an analyzer 104₁-104ₙ that loaded the reagent pack 220 corresponding to test j. The inequality used in this constraint indicates that for each requested test j, there is at least one analyzer 104₁-104ₙ (with at least one corresponding reagent pack 220 loaded) to perform the requested tests.

3) Completion Constraints: All tests ordered for the samples should be completed within the planning period. This completion constraint is directly related to the second objective function, the unmet capacity [symbol] _{MC} defined in the objective, and ensures that tests are completed as much as possible. We account for the possibility of uncompleted samples due to capacity or time-frame issues by using the slack variable ∈ⱼ for each test j. The constraint then becomes:

[symbol]

nᵢⱼ+∈ⱼ≥ [symbol] Sₐⱼ,∀j∈ [symbol] ,

withnᵢⱼ≥0,∀i∈ [symbol] ,j∈ [symbol] ,

∈ⱼ≥0,∀j∈ [symbol] .

4) Test Menu Continuity Constraints: These constraints ensures a continuous workflow of the diagnostic laboratory system 100 with an optimized workload without the need to change the fixed test menus of the analyzers 104₁-104ₙ or any move of a reagent packs 220 across analyzers 104₁-104ₙ. Such actions require additional manual effort, quality control, and analyzer calibrations and is avoided in the present optimization method. Given test menu configuration from the previous planning period, xᵢⱼ, analyzer i is not allowed to run test j in the current planning period if that test was not previously configured to run on analyzer i:

$$\{ \begin{matrix}{n}_{i j} \geq 0 , & if {x}_{ij} = 1 , \\ {n}_{i j} = 0 , & otherwise\end{matrix} .$$

5) Volume Capacity Constraint: The reagent volume required for performing the tests on an analyzer 104₁-104ₙ should be less than the total amount of volumes stored under the constant replenishment model:

nᵢⱼrᵢⱼ≤xᵢⱼVᵢⱼ,∀i∈ [symbol] ,j∈ [symbol] .

where Vᵢⱼ is the total volume of the reagent recurrently loaded on analyzer i for test j during current planning period. Consequently, we have the maximum capacity constraint from the physical limitation of the analyzer 104₁-104ₙ,

ΣⱼVᵢⱼ≤Mᵢ,∀i∈ [symbol] ,

where Mᵢ the maximum throughput of the analyzer i during current planning period.

6) Redundancy Constraint: The redundancy constraint compliments the objective function, which is useful for large-volume tests, by maximizing the total redundancy factor by explicitly enforcing a minimum number of analyzers, mⱼ, running test j:

[symbol]

I(nᵢⱼ)≥mⱼ,∀j∈ [symbol] .

The expression, I(nᵢⱼ) ∈ {0,1} indicates whether the analyzer i loads at least one reagent pack 220 for test j. The sum [symbol] I(nᵢⱼ) then accounts for the total number of analyzers 104₁-104ₙ that load the reagent pack 220 and are able to run test j. This value of mⱼ can be configured by an operator 114 of the diagnostic laboratory system 100. Use of the redundancy constraint increases the robustness of the operations of the diagnostic laboratory system 100, avoids analyzer unavailable issues, and explicitly ensures an adequate amount of analyzers to run tests having large order volumes.

7) Total Stops Constraint: The total stops constraint minimizes a number of stops in current planning period. In minimizing the number of total stops made by all the samples, the optimization method can enforce that there is at least one subset of analyzers 104₁-104ₙ to perform the tests required. We denote the set of tests requested for a sample a by:

[symbol]

ₐ={j|Sₐⱼ>0,j∈ [symbol] }

and the set of analyzers 104₁-104ₙ that has test j assigned thereto by:

[symbol]

ⱼ={i|I(nᵢⱼ)>0,i∈ [symbol] ,for a given testj}.

Then the total stops constraint can be written as follows:

[symbol]

[symbol]

Iₐᵢ≥1,∀a∈ [symbol] .

Here the term [symbol] Iₐᵢ is a non-linear combination (multiplication) of two indicator variables, I(nᵢⱼ) and Iₐᵢ, and that both are tied to the optimization variables nᵢⱼ. This non-linearity imposes additional computational complexity. In order to alleviate this, the method can relax this combination by introducing an additional binary slack variable IIₐᵢⱼ and transform the original total stop constraint into following:

[symbol]

[symbol]

IIₐᵢⱼ≥1,∀a∈ [symbol] ,

subject to:IIₐᵢⱼ≤I(nᵢⱼ),

IIₐᵢⱼ≤Iₐᵢ,

IIₐᵢⱼ≥I(nᵢⱼ)+Iₐᵢ−1,∀i∈ [symbol] ,j∈ [symbol] .

8) Minimum Analyzer Constraints: Constraints related to minimizing a number of analyzers. Minimizing the number of analyzers 104₁-104ₙ should not come at the expense of uncompleted tests such that ∈ⱼ>0. All the analyzers 104₁-104ₙ that can run test j, such that xᵢⱼ=1, should not be left out of operation if there is unmet demand for this test j. Thus, the optimization method can enforce this with the following minimum analyzer constraint:

I(∈ⱼ)xᵢⱼ≤I(nᵢⱼ),∀j∈ [symbol] ,i∈ [symbol] .

### Optimization Strategy

According to the optimization method, the optimization strategy for solving the entire problem using multiple integer linear programming (MILP) will now be described. Above we have introduced objectives and constraints with various variables that can be customized according to the size and preference for the diagnostic laboratory system 100. For example, the minimum number of analyzers 104₁-104ₙ to perform any specific assay can be configured by the laboratory operator 114 based on demand data, sample demand prediction, or the importance of that particular test. Furthermore, to reduce the workload of the operators 114 across different planning periods, the method can propose constraints on the enabled fixed test menus as well as the amount of loaded reagent packs 220 to ensure a continuous workflow.

The proposed multi-objective problem can be very challenging to solve, especially when there are potentially conflicting constraints and objectives. For example, objective 3) (Maximize Test Assignment Redundancy), described above, that promotes running specific tests on multiple analyzers, is potentially in conflict with objective 1) above, which reduces QA costs. The concept of optimality in such multi-objective problems can be characterized by Pareto optimality.

Definition 1 (Pareto-Optimal). Given k objective functions, [symbol] ₁, [symbol] ₂, . . . , [symbol] ₖ, a solution n* is Pareto-optimal if, and only if, there exists no another solution x such that [symbol] ᵢ(n)< [symbol] ᵢ(n*), ∀i ∈ {1, . . . , k}. The method can utilize two approaches in reaching Pareto optimal solutions:
1) lexicographic approach, and
2) weighted-sum approach.

The lexicographic approach is useful when a specific order of importance of the objectives may exist. The laboratory operator 114 can prioritize the order of objectives to be minimized based on the specific needs of the particular diagnostic laboratory system 100. This approach solves the multi-objective optimization problem sequentially with the objectives provided in the order of importance, while under the constraints. Given a set of ordered objectives and current planning period, the lexicographic approach proceeds, as follows:
1) Construct the optimization problem including variables and constraints associated with requirements;
2) for i=1,2, . . . , k do;
3) Minimize [symbol] ᵢ(n|x);
4) n*← the optimal solution;
5) if i<k then;
6) Add a constraint [symbol] ᵢ(n|x)≤ [symbol] ᵢ(n*|x);
7) Return n*.

The notion of (n|x) refers to the fact that solution n is conditional on the test menu configuration from the previous planning period.

The second method in finding Pareto-optimal solutions is the weighted-sum approach. Given weights w₁, w₂, . . . , wₖ ∈ [symbol] ⁺ corresponding to each objective, method solves the problem with a single objective function as following:

Minimizew₁ [symbol] _{QA}+w₂ [symbol] _{MC}−w₃ [symbol] _{R}+w₄ [symbol] _{LB}+w₅ [symbol] ₛₜₒₚₛ+w₆ [symbol] ᵢₙₛₜ

The diagnostic laboratory system 100 may use a combination of the lexicographic and weighted-sum approaches when a strict ordering of objectives does not necessarily exist. In such cases, some objectives can have the same lexicographic order and hence optimized together with associated weights. The present optimization method can employ all of these optimization approaches.

Thus, system controller 110 include a reagent pack optimization module 115, described herein that is stored in memory 113 and executed by a processor 116. The reagent pack optimization module 115 includes computer executable instructions based on the optimization method described above that may be configured and operable to receive and process input data to create a reagent pack load plan 121 that is supportive of the load plan provided by, for example, the auxiliary optimization module 117. Auxiliary optimization module 117 can provide via a separate optimization method, the fixed menus for the planning period.

Input data to be used in carrying out the optimization method in the reagent pack optimization module 115 can include the types and numbers of requested tests to be performed by diagnostic laboratory system 100, and possibly weights or priorities related to efficiency objectives that are being used.

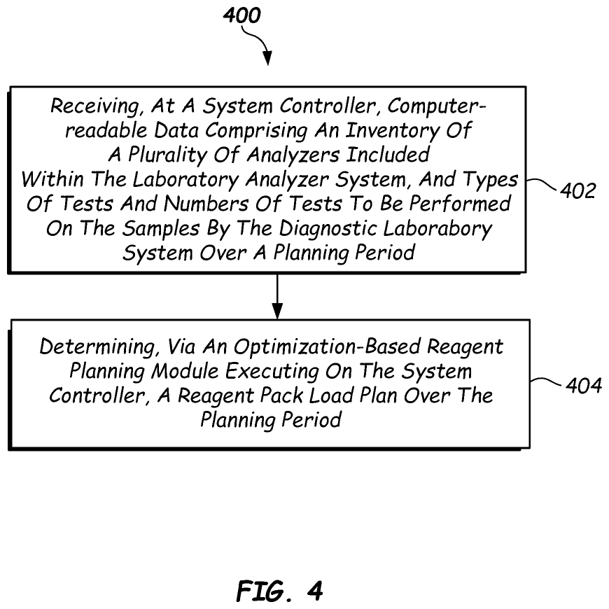

FIG. 4 illustrates a flowchart of a method 400 of optimization-based reagent pack load planning for a diagnostic laboratory system (e.g., diagnostic laboratory system 100) according to one or more embodiments of the disclosure. Method 400 may be carried out by a suitable system controller, such as, e.g., system controller 110, or other suitable computer device. Method 400 may include, at process block 402, receiving, at a system controller (e.g., system controller 110), computer-readable data comprising an inventory of a plurality of analyzers (e.g., analyzers 104₁-104ₙ) included within the diagnostic laboratory system (e.g., diagnostic laboratory system 100), and types of tests and numbers of the tests to be performed on samples by the diagnostic laboratory system over a planning period.

Method 400 may also include, in block 404, determining, via a reagent pack optimization module (e.g., reagent pack optimization module 115) executing on the system controller, a reagent pack loading plan (e.g., reagent pack loading plan 121) over the planning period. The reagent pack loading plan 121 may comprise instructions of where and what type of reagent pack 220 to load on each mounting space 216 for each of the analyzers 104₁ through 104ₙ. The reagent pack loading plan 121 may be output from the operator interface 112 in any desirable format, such as a written instruction (e.g., on paper), pictorial instruction, display on a display screen, or the like, and says which tests should be loaded onto which analyzers 104₁ through 104ₙ.

While the disclosure is susceptible to various modifications and alternative forms, specific method and system embodiments have been shown by way of example in the drawings and are described in detail herein. It should be understood, however, that the particular methods and systems disclosed herein are not intended to limit the disclosure but, to the contrary, to cover all modifications, equivalents, and alternatives falling within the scope of the claims.

What is claimed is:

1. An optimization method of a diagnostic laboratory system, comprising:
receiving, at a system controller, computer-readable data comprising an inventory of a plurality of analyzers included within the diagnostic laboratory system, and types of tests and number of the tests to be performed on samples by the diagnostic laboratory system over a planning period; and
determining, via a reagent pack optimization module executing on the system controller, a reagent pack loading plan over the planning period.

2. The optimization method of claim 1, wherein each of the plurality of analyzers has a fixed assay menu over the planning period.

3. The optimization method of claim 1, wherein the reagent pack loading plan determines optimal placement in available mounting spaces for reagent packs given the number of the tests ordered and the type of the tests to be run on the plurality of analyzers.

4. The optimization method of claim 1, wherein the optimization method utilizes demand data as an input.

5. The optimization method of claim 1, wherein the reagent pack optimization module optimizes for one or more operational efficiency considerations.

6. The optimization method of claim 5, wherein the one or more operational efficiency considerations include one or more of:
operation of the diagnostic laboratory system with a subset of the plurality of analyzers;
load balancing between the plurality of analyzers;
reduced turn-around time;
efficient reagent usage;
minimizing quality assurance costs; and
providing improved robustness of the diagnostic laboratory system.

7. The optimization method of claim 1, wherein reagent pack optimization module optimizes using one or more optimization objective functions.

8. The optimization method of claim 7, wherein one or more optimization objective functions comprise:
minimizing quality assurance costs,
minimizing unmet capacity cost,
maximizing test assignment redundancy,
optimizing workload balance,
minimizing sample visits, and
minimizing total analyzers used.

9. The optimization method of claim 8, wherein one of the one or more optimization objective functions is operated to minimize quality assurance costs.

10. The optimization method of claim 8, wherein one of the one or more optimization objective functions is operated to minimize unmet capacity cost.

11. The optimization method of claim 8, wherein one of the one or more optimization objective functions is operated to maximize test assignment redundancy.

12. The optimization method of claim 11, wherein the one of the optimization objective functions uses a redundancy factor REⱼ≥0 for each assay j.

13. The optimization method of claim 8, wherein one of the one or more optimization objective functions is operated to optimize workload balance.

14. The optimization method of claim 13, wherein optimization of the workload balance is achieved by one or more of:
operation-time balancing strives for equal processing times across all the plurality of analyzers;
test-type balancing wherein workload of each test type is distributed equally across the plurality of analyzers that have that test type deployed thereon; and
total workload balancing wherein a total number of tests to be performed should be balanced across all the plurality of analyzers.

15. The optimization method of claim 14, comprising integer, non-negative slack variables.

16. The optimization method of claim 8, wherein one of the one or more optimization objective functions is operated to minimize total analyzer visits to be made by the samples.

17. The optimization method of claim 8, wherein one of the one or more optimization objective functions is operated to minimize a total number of the analyzers used.

18. The optimization method of claim 1, wherein the optimization method comprises optimization constraints selected from a group of constraints of menu a feasibility constraint, a capacity constraint, and a workflow continuity constraint.

19. The optimization method of claim 1, comprising a menu feasibility optimization constraint that ensures that at least some of the plurality of analyzers have reagent packs loaded thereon for all the types of tests and the number of the tests to be performed on the samples by the diagnostic laboratory system over the planning period.

20. The optimization method of claim 1, comprising a capacity optimization constraint that capacity constraints arise due to physical limitations of the diagnostic laboratory system selected from a group of: a number of the plurality of analyzers, throughput of the plurality of analyzers, and a quantity of available reagent packs.

21. The method of claim 1, wherein the reagent pack optimization module comprises a mixed integer program that is optimized for operational efficiency.

22. The method of claim 1, comprising loading reagent packs on the plurality of analyzers according to the reagent pack loading plan over the planning period.

23. A diagnostic laboratory system, comprising:
a plurality of analyzers that are configured to perform tests on samples, each of the a plurality of analyzers having a fixed menu; and
a system controller coupled to the plurality of analyzers, the system controller comprising a reagent pack optimization module having computer executable instructions configured to cause the system controller to generate a reagent pack load plan for the diagnostic laboratory system over a planning period.
